(19) United States
(12) Patent Application Publication
(10) Pub. No.: US 2026/0188452 A1
(43) Pub. Date: Jul. 2, 2026

(54) Systems and Methods for Medication and Disease Management and Patient Notification

(71) Applicant: Sanofi, Paris (FR)

(72) Inventors: Niranjan TUMMINKATTI, Cambridge, MA (US); Jesse Alvarez, Cambridge, MA (US); Marjorie BEAUGAD, Cambridge, MA (US); Veronique BRUHAT, Paris (FR)

(21) Appl. No.: 19/123,132

(22) PCT Filed: Oct. 24, 2023
(86) PCT No.: PCT/IB2023/000620

§ 371 Date: Apr. 22, 2025

Related U.S. Application Data
(60) Provisional application No. 63/418,737, filed on Oct. 24, 2022.

(30) Foreign Application Priority Data
Mar. 31, 2023 (EP) 23315070.5

(51) Int. Cl. G16H 20/10; G16H 40/20

(52) U.S. Cl. CPC G16H 20/10; G16H 40/20

(57) ABSTRACT

A non-transitory computer readable storage medium includes instructions that, when executed by a computer, cause the computer to execute a program for managing a user's medical condition or treatment. The program defines a notification scheme for creating notifications associated with the user's medical condition or treatment. The system generates different types of notifications based on the occurrence of first, second, and third-level trigger events. These notifications are displayed on a graphical user interface provided by the program or an operating system of a user device. The content of the notifications can vary depending on the user's experience level. The system can also start a timer for allowing medicament in a medicament delivery device to reach room temperature in response to user interaction with a specific notification.

### CROSS REFERENCE TO RELATED APPLICATIONS

The present application is the national stage entry of International Patent Application No. PCT/IB2023/000620, filed on Oct. 24, 2023, and claims priority to Application No. EP 23315070.5, filed on Mar. 31, 2023, and U.S. Application No. 63/418,737, filed on Oct. 24, 2022, the disclosures of which are incorporated herein by reference.

### TECHNICAL FIELD

The present specification relates to systems and methods for medication and disease management and, in particular, to a computer implemented system and method for providing timely and relevant notifications to a patient to assist with management of disease progression and medication regimen.

### BACKGROUND

A significant barrier to high quality therapeutic outcomes is patient engagement and adherence with a prescribed medication regimen, as well as patient understanding of the factors affecting their disease progression and overall health.

Current systems do not effectively engage users or provide appropriate and timely messaging and reminders. Conversely, providing a user with too much information can be overwhelming and lead to user disengagement with the messaging.

### SUMMARY

Certain aspects of this disclosure relate to systems and methods which provide effective and timely messaging regarding a user's disease management and medication regimen, improve user engagement and adherence, and ultimately lead to improved therapeutic outcomes for the user.

A first aspect of this disclosure provides a non-transitory computer readable storage medium comprises instructions that, when executed by a computer, cause the computer to:
execute a program for managing a user's medical condition or treatment, the program defining a notification scheme for causing the creation of notifications associated with a user's medical condition or treatment;
when a first-level trigger event occurs, generate a silent notification configured to be displayed on a graphical user interface provided by the program for managing a user's medical condition or treatment when the user accesses the program;
when a second-level trigger event occurs, generate an indication that the user has at least one batch notification and cause the indication to be displayed; and
when a third-level trigger event occurs, generate a push notification and cause the push notification to be displayed,
   wherein the indication that the user has at least one batch notification and the push notification are caused to be displayed on a graphical user interface provided by an operating system of a user device comprising the computer.

In some embodiments, the graphical user interface provided by an operating system of a user device is a lock screen of the user device. In other embodiments, the graphical user interface provided by the program for managing a user's medical condition or treatment is a home screen of the program for managing a user's medical condition or treatment.

In some embodiments, the second-level trigger event has a higher priority than the first-level trigger event, and the third-level trigger event has a higher priority than the second-level trigger event.

In some embodiments, a first batch notification defines a threshold time for interacting with the first batch notification and, if the first batch notification is not interacted with within the threshold time, the computer is caused to generate a push notification relating to the first batch notification.

In some embodiments, the content of the silent notification and/or push notification is varied depending on the experience level of the user.

In some embodiments, a first push notification reminds a user to remove a medicament delivery device from a low temperature environment, and in response to the user interacting with the first push notification, the computer is caused to start a timer for allowing the medicament in the medicament delivery device to reach room temperature.

In some embodiments, the first, second or third-level trigger event comprises a time remaining before a next medicament administration is due.

In some embodiments, the first, second or third-level trigger event comprises a time remaining before an appointment or call.

In some embodiments, the first, second or third-level trigger event comprises a prompt to create a journal entry regarding disease symptoms and/or overall health.

In some embodiments, the first, second or third-level trigger event comprises a time remaining before a prescription period is due to end.

A second aspect of this disclosure provides a computer implemented method comprising: executing a program for managing a user's medical condition or treatment, the program defining a notification scheme for causing the creation of notifications associated with a user's medical condition or treatment;
when a first-level trigger event occurs, generating a silent notification configured to be displayed on a graphical user interface provided by the program for managing a user's medical condition or treatment when the user accesses the program;
when a second-level trigger event occurs, generating an indication that the user has at least one batch notification and cause the indication to be displayed; and
when a third-level trigger event occurs, generating a push notification and cause the push notification to be displayed,
wherein the indication that the user has at least one batch notification and the push notification are caused to be displayed on a graphical user interface provided by an operating system of a user device.

The first-level trigger event, the second-level trigger event or the third-level trigger event may each be selected from the group comprising (i) a time remaining before a next medicament administration is due, (ii) a time remaining before an appointment or call, (iii) a prompt to create a journal entry regarding disease symptoms and/or overall health and (iv) comprises a time remaining before a prescription period is due to end.

A third aspect of this disclosure provides a user device comprises processing circuitry configured to:
execute a program for managing a user's medical condition or treatment, the program defining a notification scheme for causing the creation of notifications associated with a user's medical condition or treatment;
when a first-level trigger event occurs, generate a silent notification configured to be displayed on a graphical user interface provided by the program for managing a user's medical condition or treatment when the user accesses the program;
when a second-level trigger event occurs, generate an indication that the user has at least one batch notification and cause the indication to be displayed; and
when a third-level trigger event occurs, generate a push notification and cause the push notification to be displayed, wherein the indication that the user has at least one batch notification and the push notification are caused to be displayed on a graphical user interface provided by an operating system of the user device.

### BRIEF DESCRIPTION OF THE FIGURES

So that the general concepts set out in the foregoing sections can be more fully understood, embodiments thereof will be described with reference to the accompanying drawings, in which:

FIG. 1 shows an overall architecture of a system in which the computer program operates;

FIGS. 2a to 2d show exemplary screenshots displayed by the computer program relating to silent push notifications;

FIGS. 3a and 3b show further exemplary screenshots displayed by the computer program relating to batched notifications;

FIGS. 4a and 4b show further exemplary screenshots displayed by the computer program relating to push notifications;

FIGS. 5a and 5b show first and second notification option screens;

FIG. 5c shows an injection routine control screen;

FIG. 5d shows a connected devices management screen;

FIG. 5e shows an additional notification option screen;

FIG. 6 illustrates schematically a user device for running the computer program; and

FIG. 7 is a flow chart illustrating the steps of a computer implemented method.

### DETAILED DESCRIPTION

The present disclosure relates to an application which can be run on a user device, and which assists a user with tracking of their health and disease progression and management of all aspects of their treatment. In particular, the application encourages and nudges users to maintain their treatment routine through a series of notifications on various aspects of their treatment, e.g., making journal entries, logging treatment received, generating reports prior to doctors' appointments.

The application can be used to manage a broad range of diseases which are treated by a range of different medicaments. Although some of the specific embodiments below are described in relation to the treatment of atopic dermatitis and/or asthma using a single medicament, the application is not so limited. Various other immunology indications may also be managed by the application, where one or more of these immunology indications may be treated by using a single medicament approved for use in treating the one or more immunology indications. Such a single medicament can include, for example, different dosages containing the same API, different volumes or concentrations of the same API, or different formulations containing the same API. In some embodiments, a single medicament can include an anti IL-4R mAb (e.g., Dupilumab). Further, a broad range of immunological conditions may be managed by the application. The user may be prescribed one or more drugs and the application provides personalized support for users depending on their particular drug prescription(s) and health profile. For example, the application may be configured to provide support for a number of diseases caused by Type 2 inflammation. In the context of Dermatology this may include Atopic Dermatitis, Prurigo Nodularis, Bullous Pemphigoid, an Urticaria (e.g., chronic spontaneous urticaria, or cold inducible urticaria), Hand and Foot Disease, or Pruritis, each of which may be treated by injections of Dupixent® or other injectable drugs, but also by some oral and topical medications. In the context of respiratory diseases, this may include Asthma, Nasal Polyps, Sinusitis (e.g., chronic sinusitis with or without nasal polyps), Allergic Bronchopulmonary aspergillosis (ABPA), or Allergic Fungal Rhinosinusitis (AFRS) (which may be treated by injections of Dupixent® or other injectable drugs but also by some oral medications) and Chronic Obstructive Pulmonary Disease (COPD), which may be treated by injections of Dupixent® or other injectable drugs. In the context of Gastroenterology this may include Inflammatory Bowel Disease (IBD), Eosinophilic Esophagitis (EoE), Eosinophilic Gastroenteritis (EGE), or Ulcerative Colitis, which may be treated by injections of Dupixent® or other injectable drugs but also by some oral medications.

The medicament or medicaments may be administered by injection. As used herein, the term injection or self-injection is intended to encompass intra-venous injection, intra-muscular injection, infusion, or any needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

The application can additionally assist the user in accessing treatment, including accessing insurance coverage, managing delivery of medicaments and providing education support. The application may also assist the user in managing their disease, such as by providing a tracking journal in which users and their health care providers can monitor treatment use and progress by logging quick journal entries about symptoms and medicament use and by generating reports based on the collected information. The application may also provide holistic care by providing access to learning material including instructional articles and videos and motivational articles and challenges, and by providing a connection to other connected devices, such as activity trackers and heart rate monitors to continuously update and learn about user's behavior. The application may also gather and combine information from other programs such as environmental monitoring apps or weather forecasts.

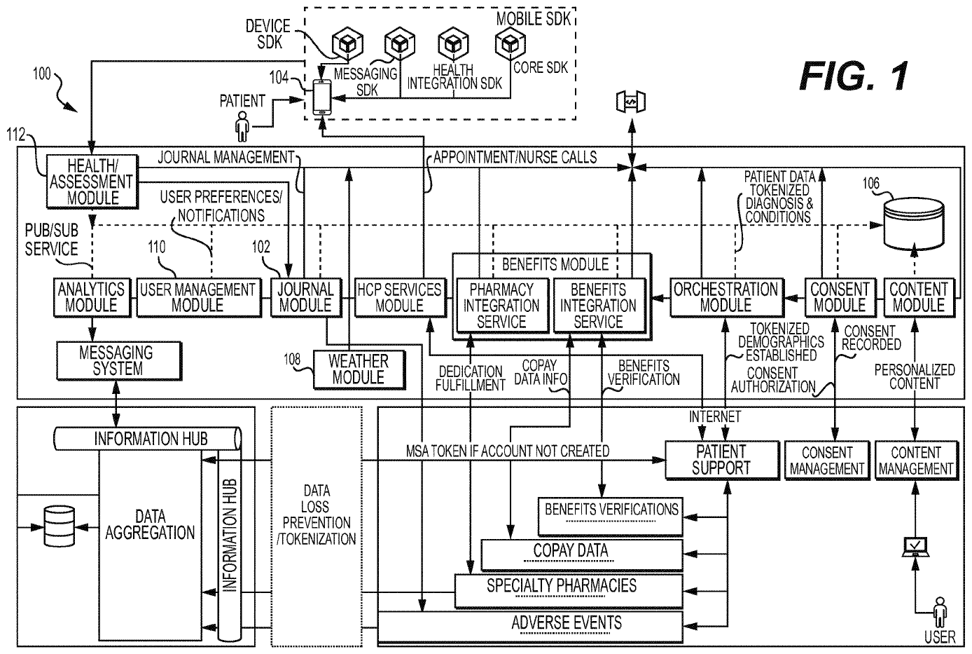

Referring to FIG. 1, an overall architecture 100 for a disease management and medication regimen management system is shown. The system architecture 100 illustrates a number of functional modules and the data links between these. The system architecture 100 comprises a user device 104 running an application with a number of functional modules, illustrated in the central box. The architecture 100 also illustrates how the application run by the user device 104 can interact with other service providers to enhance the information which can be provided to the user through the application. For instance, by communicating with insurance/benefit provider systems and pharmacies.

One of the functional modules is a journal module 102. The journal module 102 is configured to allow a user to maintain a journal relating to one or more medical conditions of the user. The journal may comprise a plurality of journal entries that detail episodes of the one or more medical conditions, such as symptoms, user specific health data, contextual information, or the like. The journal may be used to monitor the one or more medical conditions for patterns, and to identify potential triggers for episodes of the one or more medical conditions. The journal module 102 may communicate with an external service called adverse events. The journal module may transmit patient information associated with episodes of the one or more medical conditions to the adverse events service.

The journal may further maintain a record of the adherence of the user to a treatment regimen, such as a medicament regime. For instance, the journal entry can include a record logging a dose of medicament (e.g. a scheduled dose) that has been taken by the user, such as an injection log. The record can form a dose record including a variety of information associated with the dose administered by the user. The record can be stored as part of the journal entry or independently from the journal entry.

The functional module may further comprise a memory module, or database 106. The memory module 106 is configured to store journal entries made by the user via the journal module 102 for later retrieval and/or analysis. The memory module 106 may, in some embodiments, be linked to the cloud, and store journal entries remotely.

In some embodiments, the functional modules may further comprise a weather module 108. The weather module 108 is configured to determine and/or record weather conditions at the location of the user. The user location can be determined, for example, by a GPS capability of the user device. The weather module 108 may, for example, access an online weather forecasting system to obtain current weather conditions, previous weather conditions and/or predicted future weather conditions. The weather module 108 may provide weather conditions to the journal module 102 for inclusion in journal entries. As well as weather conditions such as general weather condition, temperature, humidity, air pressure and UV index, the weather module may additionally obtain air quality information such as pollen count, pollution index, NO₂ count and/or an air quality index.

One of these functional modules is a user management module 110 which is responsible for obtaining user preferences and for generating notifications for output on the user device 104. Necessary data for the notification scheme may be stored on a local database 106 of the user device 104. The user management module 110 may also update the information held in the database 106 as the application is used. The architecture 100 also illustrates how the application run by the user device 104 can interact with other service providers to enhance the information which can be provided to the user through the application.

The user management module 110 may define and help implement a notification scheme which determines when a notification should be generated and how it should be output on the user device 104. The notification scheme has three main notification types; (i) a silent notification; (ii) a batched summary notification; and (iii) a push notification.

The silent notification may be pushed into the application silently. The user will be presented with the silent notification within the app, for example on an app home screen. In some embodiments, the silent notifications are surfaced to the user when certain requirements are met and do not require any action on user's part. The batched summary notification may relate to several different reminders and other notifications. The user can interact with the batched summary notification to expand and show the individual notifications. The batch summary notification indicates the number of notifications which are present for the user to review. The push notification may relate to a single notification and is pushed to the lock screen or home screen of the user device 104. The push notifications are time-sensitive and actionable items that require the user's immediate attention. They are reserved for actions related to the user's treatment routine or as a follow up to actions that can impact the user's access to the treatment/drug.

Notifications can escalate, i.e. move up the notification types, depending on the user's action (or lack thereof). For example:
a. When the user is 5 days away from their next medicament dose, their next dosing reminder will show up as a silent notification in the application, for example underneath the carousel on the App homepage.
b. In the days leading up to the dosing, the application will push batch notifications to the user to either review instructional videos, book a call with a nurse, or review the instructions for self-administration.
c. On the day of the dosing and an hour prior to the scheduled time, the application will push a notification to the user to take their dose. If the user's medication is kept in the fridge, the notification will instruct the user to take their medicament out of the fridge. Interacting with this notification will trigger a warm-up timer, set to their medication dose.

Another of the functional modules is a Health/Assessment Module 112. The Health/Assessment Module 112 is responsible for onboarding the user and obtaining details regarding the user's medical conditions and medicaments being used to treat these. The Health/Assessment Module 112 may also gather information about the user's prescriptions and manage delivery of medicaments.

Another of the functional modules is a Health Care Professional (HCP) Services Module. The HCP Services Module is responsible for facilitating contact with a nurse, doctor or other health care professional, including scheduling reminders relating to appointments and calls, prompting the user to create reports or otherwise prepare for an upcoming appointment or call, initiating voice or video calls, providing follow-up notifications after calls or appointments and initiating a journal entry after calls or appointments. The HCP Services Module interacts with an internet-based Patient Support Program. Health care professionals may also have access to certain aspects and information held by the Patient Support Program, in order to facilitate contact with the user and monitor the user's treatment and/or disease progression.

Another of these functional modules is a Consent Module (also referred to as a consent management module) which, in conjunction with other aspects of the system, is responsible for managing consents and permissions for accessing patient specific information, such as health related data for the user. The Memory Module 106 may be configured to store user ID and role information as well as consent templates for use by the Consent Module. The Memory Module 106 may also store consent and permission information input by the user via the consent Module, including any time limits set on the consents and permissions.

The Consent Module may communicate with an external service called Consent Management service. The consent and permission information generated by the Consent Module may be stored or copied to the Consent Management service. The Consent Management service may also be responsible for sending the patient specific information to third parties when the associated consents indicate that this is permitted.

Another of the functions modules is a Content Module. The content module is responsible for receiving and recording personalised content and/or preferences set by the user. The content module 124 may communicate with an external service called Content Management. The content management service responsible for providing patient specific content or settings from an external user, such as a healthcare professional. The content module may interact with the memory module 106 to store the personalised content and/or settings.

Another of the functional modules is a Benefits Module which, in conjunction with other aspects of the system, is responsible for managing patient access to health care. The Benefits Module may include a pharmacy integration service and/or a benefits integration service. The information generated by the benefits module may be responsible for sending patient specific information to third parties to indicate patient access to health care services. The benefits module may interact with external services, such as benefit provider systems and pharmacy services.

Another of the functional modules is an Analytics Module. The analytics module is responsible for analysing the patient information associated with the status and/or treatment of the user's medical conditions.

Another of the functional modules is a Messaging System. The Messaging System may communicate with the analytics module. For instance, if the analytics module identifies an episode or a trigger to an episode of the one or more medical conditioners of the user, the messaging system outputs a message communicating this result. The messaging system may communicate with an external service called an Information Hub. The information hub may store the data received from the messaging system. The information hub may also interact with other service providers to enhance the information which can be stored in respect of the user through the application. For instance, by communicating with the patient support program, insurance/benefit provider systems and pharmacies, adverse events services and so on.

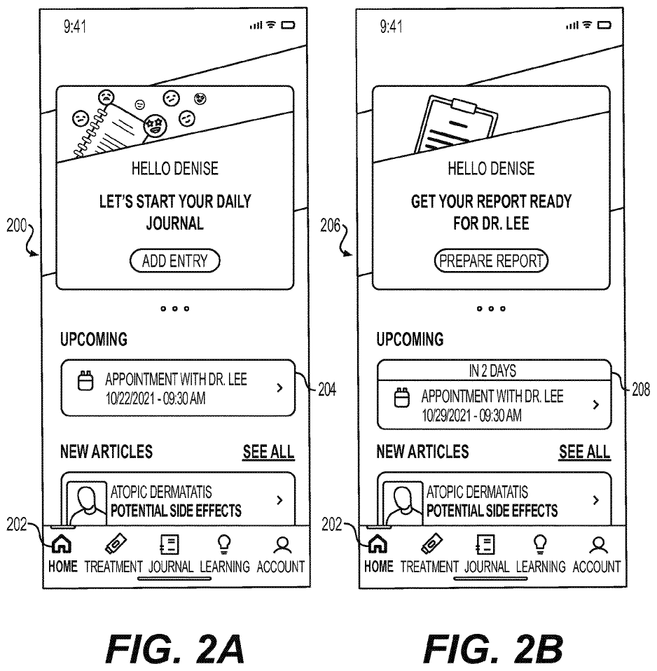

Referring now to FIGS. 2a to 2d, exemplary screenshots displayed by the application are shown. The screenshots in FIGS. 2a to 2d relate to silent notification, which are displayed only within the application.

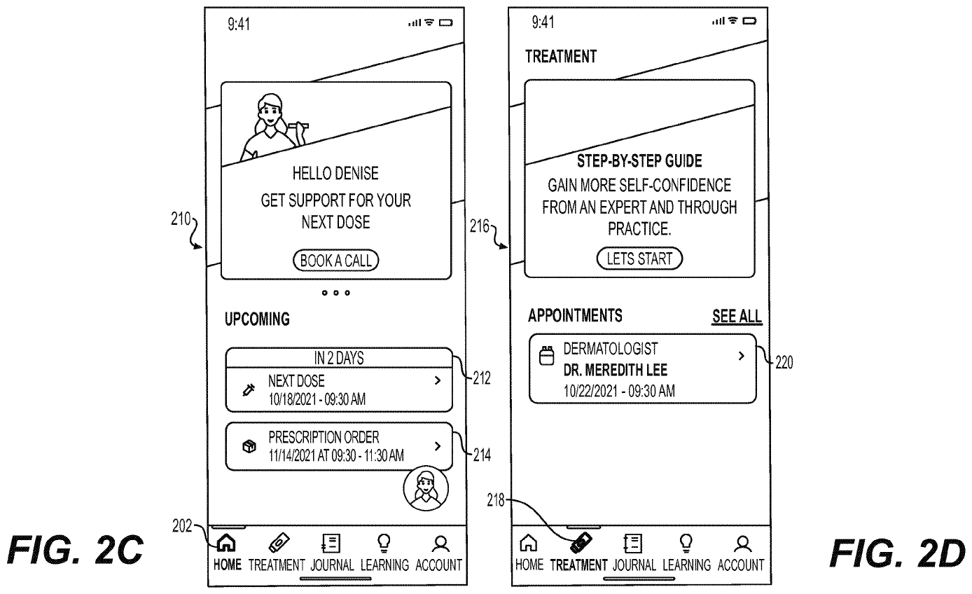

In the first screenshot 200 shown in FIG. 2a, the home screen tab 202 is selected. The application shows a number of user selectable tabs at the bottom of the screen, the tabs including at least two of Home screen, Treatment, Journal, Learning and Account (or Settings). The home screen has a notification area where silent notifications are shown. A first silent notification 204 is shown reminding the user that they have a doctor's appointment and indicating the date and time of the appointment. Interacting with the first silent notification 204 may open the notification center (see FIG. 3b), where any further details relating to the notification can be seen as well as any other notifications which are awaiting the user's review. In the top portion of the home screen a graphic with a suggested action is displayed. The content of the suggested action changes depending on the proximity of a user's next dosing/injection, doctor's appointment, prescription renewal/delivery date etc. In the example of the first screenshot 200, since the user's doctor's appointment is more than a few days away (for example more than three days away), the suggested action does not relate to the appointment, but to a daily journal entry.

In the second screenshot 206 shown in FIG. 2b, the home screen tab 202 is again shown. A second silent notification 208 is shown reminding the user that they have a doctor's appointment and indicating the date and time of the appointment. Since the doctor's appointment is only two days away, the time remaining before the appointment is more prominently displayed as part of the second silent notification 208. Also, the suggested action displayed in the top portion of the home screen relates to the upcoming doctor's appointment, in particular a reminder to prepare a report for the doctor to review at the appointment. In this manner, the application provides pertinent and timely notifications and associated suggested actions to the user.

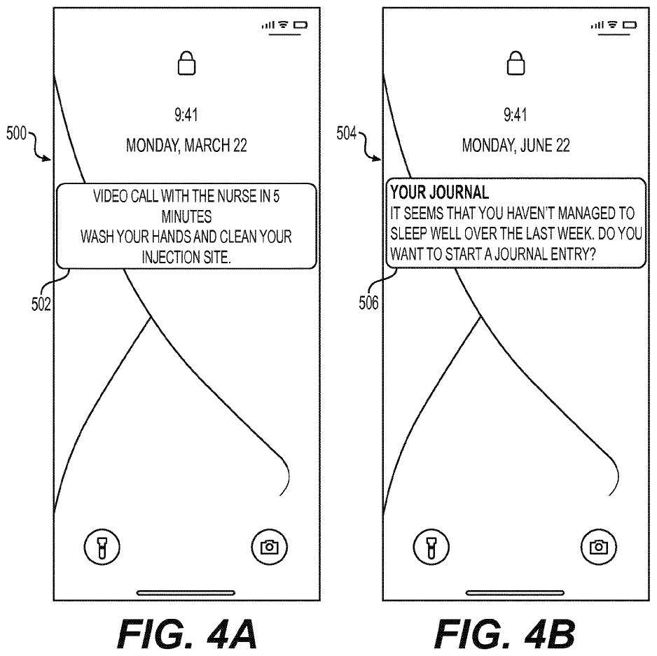

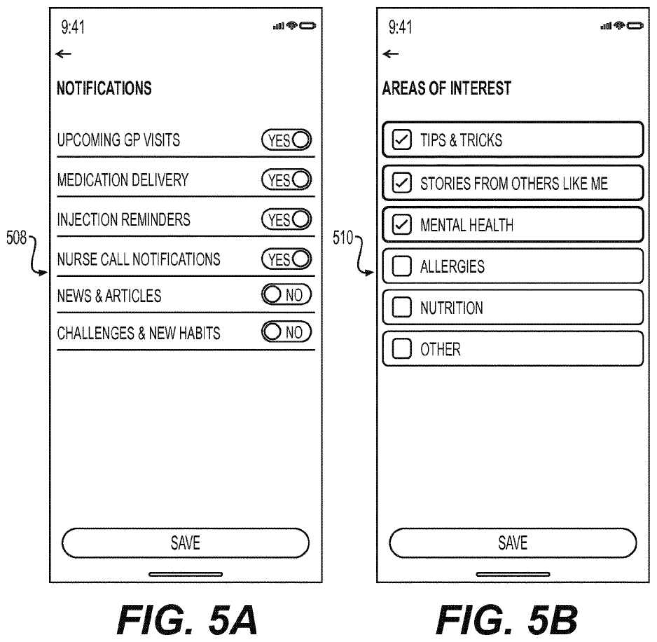

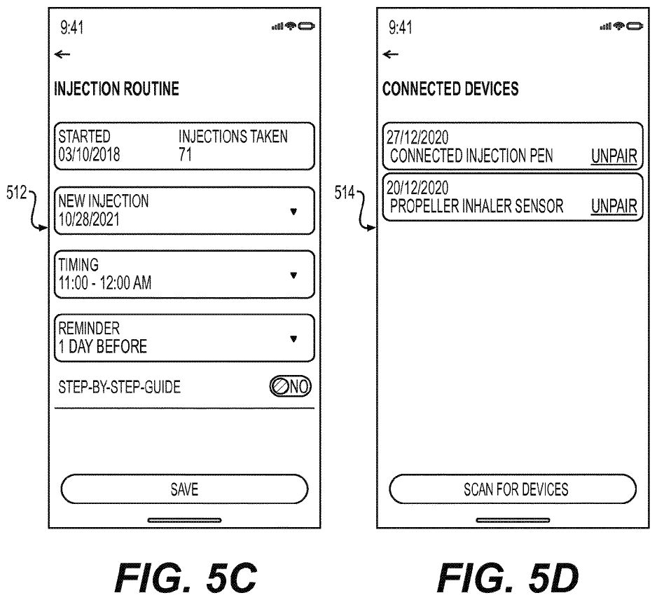

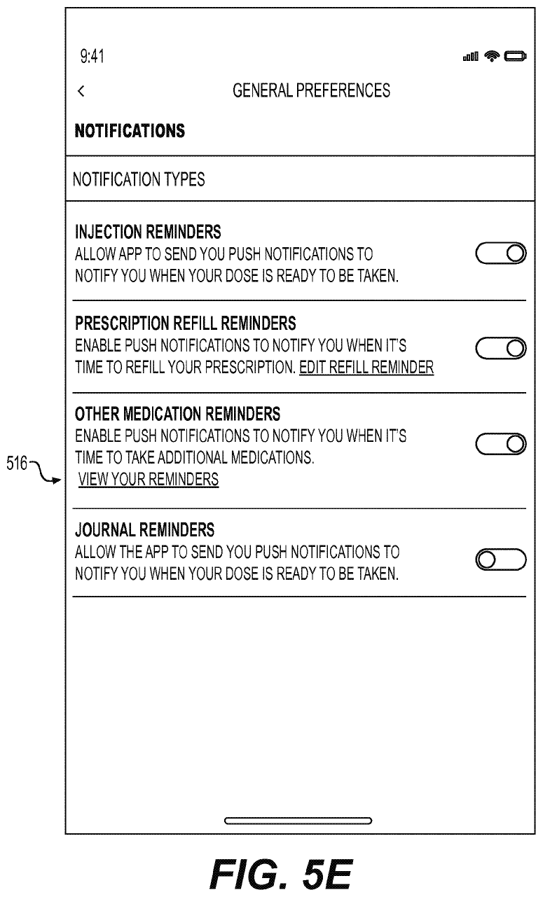

In the third screenshot 210 shown in FIG. 2c, the home screen tab 202 is again shown. In this example a third silent notification 212 and a fourth silent notification 214 are shown in the notification area of the home screen. These are of a different type from the previously described first and second silent notifications. The third silent notification 212 relates to the scheduled time of the user's next medicament dose. The date and time of the scheduled dose are indicated in the third silent notification 212, however these details may have been set by the user (see FIG. 5c). Since the time remaining before the scheduled dosing event is only two days away, the time remaining is more prominently displayed at the top of the third silent notification 212. As the notification relates to a schedule medicament administration, the suggested action displayed in the top portion of the home screen relates to supporting the user with administering the medicament. In particular, the suggested action may be to book a call with a healthcare professional.

The fourth silent notification 214 relates to an upcoming delivery of medicament. The fourth silent notification 214 indicates the date and a time window for the delivery. Interacting with either the third silent notification 212 or the fourth silent notification 214 may open the notification center (see FIG. 3b), where any further details relating to the notification can be seen as well as any other notifications which are awaiting the user's review.

Some silent notifications may be displayed in others of the user selectable tabs at the bottom of the screen. In the fourth screen shot 216 shown in FIG. 2d, the treatment tab 218 is selected. The treatment screen also has a notification area where silent notifications are shown, but a more limited range of notification may appear here, compared to the home screen. For example only notifications relating to doctor's appointment and scheduled dosing events may be shown in the treatment screen. A fifth silent notification 220 is shown reminding the user that they have a doctor's appointment and indicating the date and time of the appointment. Interacting with the fifth silent notification 220 may open the notification center (see FIG. 3b), where any further details relating to the notification can be seen as well as any other notifications which are awaiting the user's review. Similarly to the home screen, the treatment screen also devotes the top portion of the treatment screen to displaying a graphic with a suggested action. The suggested action displayed in the top portion of the treatment screen relates to supporting the user with administering the medicament. In particular, the suggested action is a link to information and/or instruction videos for self-administration of the medicament.

The silent notifications described with respect to FIGS. 2a to 2d are merely used as examples of the sort of information which can be communicated to the user by silent notification. The silent notifications may be displayed in order of decreasing urgency. For example, if the most urgent action is the user's next scheduled dose, then this reminder may appear first in the list of silent notifications, or may be the only silent notification. The content of the silent notification may also depend on the particular tab which is being shown. For example, when the home tab 202 is selected, the most urgent silent notification may be displayed. When the journal tab is selected, a notification relating to the user's next appointment may be displayed even if this is not the most urgent notification. When the treatments tab is selected, a notification relating to the user's next scheduled dose may be displayed even if this is not the most urgent notification. A fuller, but not exhaustive, list of notifications which may be provided to the user by silent notification is presented below in Table 1.

In the notification center, options to alter some aspect of the notification, such as the time of an appointment or scheduled dosing event, may be accessible. Where the notification relates to a delivery of medicament, a link to contact the carrier/shipping company may be provided so that the day and time of delivery may be altered.

Referring now to FIGS. 3a and 3b, further exemplary screenshots are shown. The screenshots in FIGS. 3a and 3b relate to batched notifications. The fifth screenshot 300 shown in FIG. 3a is of the lock screen of the user device running the application. The lock screen displays a batch notification 302 indicating that the user has a number of notifications from the application awaiting their review. In this manner, the user is not overwhelmed with many separate notifications, where these notifications are not time critical or do not require immediate user action. The user is presented with useful information about the number and source of the notifications and can then choose when to interact with the batch notification 302. In the example of the fifth screenshot 300 shown in FIG. 3a, the user has three notifications awaiting their review.

Interacting with the batch notification 302 may open the notification center as shown in the sixth screenshot 304, shown in FIG. 3b. The notifications are there shown individually. The user can interact with any of the displayed notifications in the notification center to navigate to a new page where appropriate action can be carried out.

The batched notifications described with respect to FIGS. 3a and 3b are merely used as examples of the sort of information which can be communicated to the user by batch notification. A fuller, but not exhaustive, list of notifications which may be provided to the user by batch notification is presented below in Tables 1 and 2.

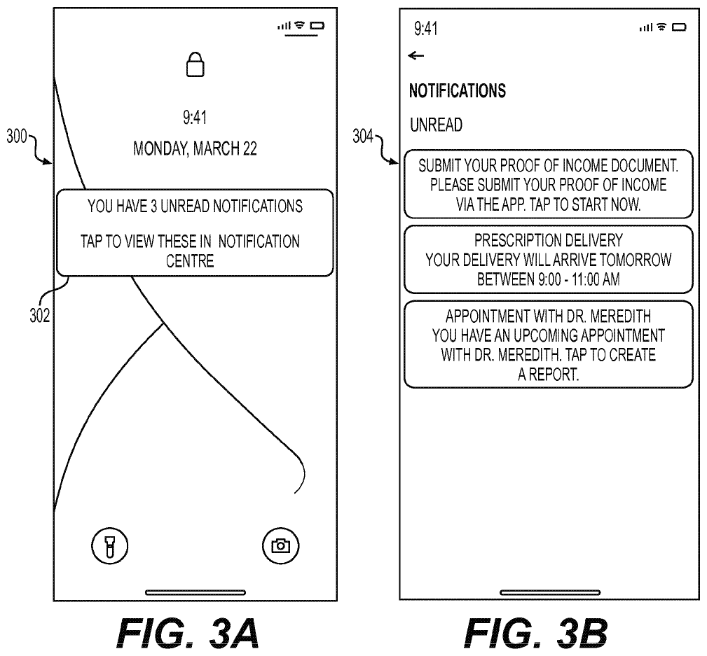

Referring now to FIGS. 4a and 4b, further exemplary screenshots are shown. The screenshots in FIGS. 4a and 4b relate to push notifications. The seventh screenshot 500 shown in FIG. 4a is of the lock screen of the user device running the application. The lock screen displays a push notification 502 indicating that the user has a call with a nurse in 5 minutes time and containing some instructions for preparing for the call. The time remaining before an event displayed as a push notification may be actively updated. For example, a notification of an upcoming call with a nurse may be displayed 1 hour before the call is due to start. The displayed time until the call may then be updated, for example in increments of 5 minutes. Interacting with the push notification 502 may navigate the user to information to assist them in preparing for an injection, or to recent journal entries relating to their health.

As can be appreciated, the push notification 502 is of a time sensitive nature and requires action by the user. In this manner, urgent or time sensitive tasks are prioritized by the notification system and the user learns to trust that the application will only display a push notification for such time sensitive tasks. The user is therefore less likely to ignore any such notification and to be better prepared for dosing events and appointments and more likely to adhere to their prescribed dosing regimen. The appearance of the push notification may also be accompanied by an audible and/or haptic output on the user's device.

The eighth screenshot 504 shown in FIG. 4b illustrates a further example of a push notification 506. In this example, the push notification 506 is a prompt to complete a journal entry. In particular, the application has received information that the user did not sleep well, for example from an external monitoring device, or via another application running on the user's device which shares information with the application. Interacting with the push notification 506 may navigate the user to a journal entry start page within the application.

Display of this notification in a timely manner can prompt the user to make a journal entry, which is a time sensitive task. By prompting the user to make a journal entry soon after the user has experienced a symptom, for example, is likely to result in a journal entry that is more accurate given that memories relating to acute events may fade over time. In this manner, the user is prompted to make useful records relating to their disease progression and overall health, providing more and better-quality information for Healthcare professionals.

The push notifications described with respect to FIGS. 4a and 4b are merely used as examples of the sort of information which can be communicated to the user by push notification. A fuller, but not exhaustive, list of notifications which may be provided to the user by push notification is presented below in Tables 1 and 2.

The application may provide the user with various options for customizing the type of notifications they receive and the timing of any reminders to take their medication. FIG. 5a shows a first notification option screen 508. The first notification option screen 508 has a number of user selectable elements which allow the user to specify whether they want to be notified about particular events. The list of events may comprise upcoming doctor's appointments, medication delivery, dosing event reminders such as injection reminders, nurse call notifications, news and articles and challenges and new habits. FIG. 5b shows a second notification option screen 510 which allows the user to select areas of interest. The user may be presented with notifications related to the selected areas of interest. Allowing the user to specify which notifications they will receive and which areas they are interested in hearing about improves the level of customization offered by the notification system and improves user engagement and satisfaction.

FIG. 5c shows an injection routine control screen 512. The injection routine control screen 512 allows the user to specify at what time of day they usually perform their injection, so that they can be reminded at an appropriate time. The user may also use this screen to specify how long before the scheduled injection time they wish to be reminded about the injection, for example 1 day before. There is also a selectable option for whether to provide a step-by-step guide as part of the injection reminder. This option may be toggled to ‘yes’ by default until the user specifies otherwise. Although this screen is described in terms of an injection reminder, the application is also able to assists the user in managing other forms of medication administration, such as oral and topical medicaments.

The timing of the notifications, in particular the push notification and batch notification may initially be defined by the application. However, the application may also dynamically adjust the timing of the notifications as the user engages with the application. The application can learn about particular user behaviors and can track when the user is most active and when they are most likely to open the application. The application can then adjust the timing of notifications accordingly for maximum impact and maximum user engagement.

FIG. 5d shows a connected devices management screen 514. This screen lists any connected devices capable of wireless communication and associated with the user. The user can use this screen to pair a new device, such as an injector or an inhaler, with the application for managing the user's medical condition or treatment, or remove any unwanted devices.

FIG. 5e shows an additional notification option screen 516. The additional notification option screen 516 may be displayed by the application instead of or in addition to the notification option screen 508 of FIG. 5a. The alternative notification option screen 516 has a different list of notification types which the user can enable or disable. The list of notifications may comprise dosing event reminders such as injection reminders, prescription refill reminders, other medication reminders (where the user has indicated in the application which other medicaments they are taking and on what schedule) and journal reminders. Some of the notification options on the alternative notification option screen 516 may have links which the user can select to edit the various related reminders which are set in the application, or to view upcoming reminders.

Table 1 and Table 2 below detail a range of different notifications which may be generated by the notification system described herein. The logic column specifies the conditions required for a particular notification to be generated. This may take the form of a predefined time before an event, at the time of an event or at a predefined time after an event. In some other examples, the logic conditions require that the user has or has not completed a particular task in addition to the time constraints.

The copy for early users column specifies the text which will be displayed as part of the notification for inexperienced users. The copy for experienced users column specifies the text which will be displayed as part of the notification for experienced users. An inexperience user may be defined as a user who has completed fewer than a predefined number of injections/medicament administrations, or who has been using the application for less than a predetermined period of time. In some other embodiments the user may have the option to define whether they are experienced or inexperienced.

The Notification Type column indicates the priority level of the notification. Any event which triggers a silent push notification is a first-level trigger event. Any event which triggers a Batched notification is a second-level trigger event. Any event which triggers a push notification is a third-level trigger event. Some events begin at a particular level and can escalate to a higher level if the user does not interact with the notification or complete the task that the notification relates to within a given time period (see for example Document 01-10 in Table 2).

The Notification Point column indicates where the notification can or might be displayed. This will usually depend on the notification type. Push notifications may be displayed on the lock screen of the user device. Silent push notifications are displayed somewhere within the application, or at multiple places within the application. For example, silent notifications may appear on the Homepage/homescreen of the application. The homepage of the application may have a defined area for the display of notification, or several different areas in which a notification might appear. These areas may be referred to as cards (e.g., injection card, carousel card). In some other embodiments, the injection card and carousel card are separate pages within the application. If the silent notifications relate to the user's treatment, it may also appear in the treatment page of the application.

Batched notifications appear individually in the notification center. In this instance, a notification that one or more batched notifications are awaiting the user's review also appears on the lock screen of the user device, as previously described. In addition, the push notifications may also appear individually in the notification center. The silent notifications may optionally appear in the notification center.

Where a particular notification can escalate up the priority levels, only the initial location of display of the notification is indicated. The application and notification scheme are applicable to a wide range of different medicaments, which may be administered via injection, orally or topically and may relate to a wide range of different diseases and medical conditions. The application and notification scheme are also able to notify the user in relation to several different medical conditions and treatment regimen simultaneously. In some embodiments, several difference instances of the notification scheme may run on the application, relating to each of the medical conditions being managed or to each of the medicaments being administered.

The priority level of a notification may also be based on the medical conditions that the particular user has. For example, some notifications may have a higher priority (and for example displayed as a push notification) if the user has indicated that they have a first medical condition. The first medical condition may be Asthma. The same notification may be given a lower priority, and for example displayed as a batch notification or silent push if the user has indicated that they have a second medical condition, but not the first medical condition. The second medical condition may be Atopic Dermatitis.

TABLE 1

| | Notification | | Copy for | Copy for | Notification | Notification |
|---|---|---|---|---|---|---|
| Purpose | name | Logic | early users | experienced users | point | Type |
| Treatment: | Treatment | 5 days | Your next [drug | Your next [drug | Homepage, | Silent |
| Next | 01 | prior to | name] dose | name] dose | injection | push |
| injection | | next | (Date/time) | (Date/time) | card | |
| | | injection | | | | |
| | Treatment | 4 days | Title: | (N/A?) User won't | Notification | Batched |
| | 02 | prior to | Your [drug name] | need an | center | |
| | | next | dose is in 2 days | appointment to | | |
| | | injection (if | Body: | help with dose | | |
| | | weekend falls | Book a call with a | Title: | | |
| | | within this | nurse who will | Your [drug name] | | |
| | | period), 2 | guide you with | dose is in 2 days | | |
| | | days prior | your next dose. | Body: | | |
| | | otherwise | | Questions about | | |
| | | e.g. if | | your [drug name] | | |
| | | injection is | | treatment? Book | | |
| | | on Wed, | | a call with a | | |
| | | notification | | nurse. | | |
| | | comes up | | | | |
| | | on Mon, if | | | | |
| | | injection is | | | | |
| | | on Sat, | | | | |
| | | notification | | | | |
| | | comes up | | | | |
| | | on Wed | | | | |
| | Treatment | 4 days | Title: | Title: | Notification | Batched |
| | 03 | prior to | Your [drug name] | Want extra help | center | |
| | | next | dose is in 2 days | with your | | |
| | | injection | Body: | insurance or | | |
| | | (if weekend | Get free support | [drug name] | | |
| | | falls | with a nurse by | deliveries? | | |
| | | within this | signing up with a | Body: | | |
| | | period), 2 | Patient Support | Sign up to a | | |
| | | days prior | Program | Patient Support | | |
| | | otherwise | | Program to get | | |
| | | | | free access to | | |
| | | | | these services | | |
| | Treatment | 2 days | Title: | Title: | Notification | Batched |
| | 04 | prior to | Your [drug name] | Your [drug name] | center | |
| | | next | dose is in 2 days | dose is in 2 days | | |
| | | injection, | Body: | Body: | | |
| | | injection | Open the app to | Need a | | |
| | | guide | review the | refresher? | | |
| | | | injection guide. | Review the | | |
| | | | | injection guide. | | |
| | Treatment | 2 days | Title: | Title: | Notification | Batched |
| | 05 | prior to | Your [drug name] | Your [drug name] | center | |
| | | next | dose is in 2 days | dose is in 2 days | | |
| | | injection, | Body: | Body: | | |
| | | instructional | Need a quick | Need a quick | | |
| | | video | refresher on how | refresher on how | | |
| | | | to inject? Watch | to inject? Watch | | |
| | | | the instructional | the instructional | | |
| | | | video | video | | |
| Treatment: | Treatment | Reminder | Title: | Title: | Lock | Push |
| Day of | 06 | one hour | Time to take your | Your [drug name] | screen | notification |
| injection | | prior to | [drug name] out | injection is today | | |
| | | scheduled | of the fridge | Body: | | |
| | | injection to | Body: | Time to take your | | |
| | | take [drug | You'll need to let | [drug name] out | | |
| | | name] of | it get up to room | of the fridge and | | |
| | | the fridge | temperature | start the timer. | | |
| | | | before you inject | | | |
| | Treatment | When | Title: | Title: | Lock | Push |
| | 07 | [drug | Your [drug name] | Your [drug name] | screen | notification |
| | | name] is | is ready now | is ready now | | |
| | | ready, after | Body: | Body: | | |
| | | timer runs | Don't worry, we'll | Tap to complete | | |
| | | out | guide you | your treatment. | | |
| | | | through the steps | | | |
| | | | to properly inject | | | |
| | | | your [drug name]. | | | |
| | Treatment | At | Title: | Title: | Lock | Push |
| | 08 | scheduled | Forgot to take | Forgot to take | screen | notification |
| | | time of | your [drug name] | your [drug name] | | |
| | | injection, | out of the fridge? | out of the fridge? | | |
| | | when user | Body: | Body: | | |
| | | hasn't | We sent you a | Confirm you have | | |
| | | responded | notification an | taken [drug | | |
| | | to take | hour ago to take | name] out of the | | |
| | | [drug | your [drug name] | fridge | | |
| | | name] out | out of the fridge. | | | |
| | | of the | We want to help | | | |
| | | fridge | you get better | | | |
| | | (Treatment | management of | | | |
| | | 06) | your [asthma/ | | | |
| | | | atopic dermatitis]. | | | |
| | Treatment | If user | Title: | Title: | Lock | Push |
| | 09 | hasn't | Have you taken | Don't forget to log | screen | notification |
| | | logged | your [drug name] | your dose for | | |
| | | their | today? | today. | | |
| | | scheduled | Body: | Body: | | |
| | | injection, | You were due for | Open the app to | | |
| | | surface up | your injection at | confirm you have | | |
| | | 1 hour after | [8:30am] today. | taken your dose. | | |
| | | scheduled | | | | |
| | | time of | | | | |
| | | injection | | | | |
| Treatment: | Missed | If user is | Title: | Title: | Lock | Push |
| Missed | 01 | late for | Did you miss | Did you miss | screen | notification |
| Injection | | injection, | your last dose? | your last dose? | | |
| | | 1-7 days | Body: | Body: | | |
| | | Sent to | Hey [Denise], | Open the app to | | |
| | | user once | looks like you | confirm you have | | |
| | | every day | missed your last | taken your dose. | | |
| | | | scheduled dose | | | |
| | | | on June 1. | | | |
| | Missed | User is 8+ | Title: | Title: | Lock | Push |
| | 02 | days late | Looks like you're | You've missed | screen | notification |
| | | for | more than 7 days | your last dose by | | |
| | | injection, | late with your last | more than 7 days | | |
| | | on a two | dose | Body: | | |
| | | week cycle | Body: | We recommend | | |
| | | | We recommend | that you wait until | | |
| | | | that you wait until | your next | | |
| | | | your next | scheduled dose | | |
| | | | scheduled dose | on June 15 | | |
| | | | on June 15 | | | |
| | Missed | User is 8+ | Title: | Title: | Lock | Push |
| | 03 | days late | Looks like you're | You've missed | screen | notification |
| | | for | more than 7 days | your last dose by | | |
| | | injection, | late with your last | more than 7 days | | |
| | | on a four | dose | Body: | | |
| | | week cycle | Body: | Your treatment | | |
| | | | When you take | schedule will be | | |
| | | | and log your next | adjusted based | | |
| | | | dose, we'll | on when you take | | |
| | | | readjust your | and log your next | | |
| | | | treatment | dose. | | |
| | | | schedule based | | | |
| | | | on your injection | | | |
| | | | date as per our | | | |
| | | | recommended | | | |
| | | | guidance. | | | |
| Appointment: | Appointment | If user | Title: | Title: | Homepage, | Silent |
| Add | 01 | doesn't | Do you have an | Book a call with | carousel | push |
| appointment | | have an | appointment | your nurse | card | |
| | | appointment | booked with your | Body: | | |
| | | added in | nurse? | You can contact | | |
| | | the app | Body: | a nurse for | | |
| | | | You can contact | support with your | | |
| | | | a nurse for | treatment. | | |
| | | | support with your | | | |
| | | | treatment. | | | |
| Appointment: | Appointment | 3 days | Title: | Title: | Homepage + | Silent |
| Prepare | 02 | prior to | Let's get your | Get your report | Treatment, | push |
| report | | doctor | report ready for | ready for your | carousel | |
| | | appointment | your appointment | appointment Dr. | card/card | |
| | | | in 3 days | Lee | | |
| | | | Body: | Body: | | |
| | | | Gather your | Gather your | | |
| | | | journal entries | journal entries | | |
| | | | and quality of life | and quality of life | | |
| | | | questionnaires | questionnaires. | | |
| | | | ready for your | | | |
| | | | appointment with | | | |
| | | | Dr. Lee. | | | |
| | Appointment | 2 days | Title: | Title: | Notification | Batched |
| | 03 | prior to | Let's get your | Get your report | center | |
| | | doctor | report ready for | ready for your | | |
| | | appointment, | your appointment | appointment Dr. | | |
| | | if user | in 2 days | Lee | | |
| | | hasn't | Body: | Body: | | |
| | | generated | Gather your | Gather your | | |
| | | a report | journal entries | journal entries | | |
| | | | and quality of life | and quality of life | | |
| | | | questionnaires | questionnaires. | | |
| | | | ready for your | | | |
| | | | appointment with | | | |
| | | | Dr. Lee. | | | |
| | Appointment | 1 day prior | Title: | Title: | Notification | Push |
| | 04 | to doctor | Let's get your | Get your report | center | notification |
| | | appointment, | report ready for | ready for your | | |
| | | if user | your appointment | appointment Dr. | | |
| | | hasn't | in 1 days | Lee | | |
| | | generated | Body: | Body: | | |
| | | a report | Gather your | Gather your | | |
| | | | journal entries | journal entries | | |
| | | | and quality of life | and quality of life | | |
| | | | questionnaires | questionnaires. | | |
| | | | ready for your | | | |
| | | | appointment with | | | |
| | | | Dr. Lee. | | | |
| | Appointment | Day of | Title: | Title: | Notification | Push |
| | 05 | doctor | Let's get your | Get your report | center | notification |
| | | appointment, | report ready for | ready for your | | |
| | | if user | your appointment | appointment Dr. | | |
| | | hasn't | today | Lee | | |
| | | generated | Body: | Body: | | |
| | | a report | Gather your | Gather your | | |
| | | | journal entries | journal entries | | |
| | | | and quality of life | and quality of life | | |
| | | | questionnaires | questionnaires. | | |
| | | | ready to share | | | |
| | | | with your doctor. | | | |
| Appointment: | Appointment | View your | Title: | Title: | Homepage, | |
| View | 06 | report | Review your | See your latest | carousel | |
| report | | | reports at any | report | card | |
| | | | time | Body: | | |
| | | | Body: | View your report | | |
| | | | Access your | and share it with | | |
| | | | reports you've | your doctor for | | |
| | | | generated and | your upcoming | | |
| | | | easily share them | appointment. | | |
| | | | with your doctor. | | | |
| Journal: | Journal | Carouse | Title: | Title: | Homepage, | Silent |
| Add entry | 01 | card to | Log entries to | Don't forget | carousel | push |
| | | encourage | monitor and track | adding to your | card | |
| | | users to | your condition | journal will give | | |
| | | start | Body: | insights on your | | |
| | | adding to | The more entries | condition | | |
| | | their | you add, you'll | Body: | | |
| | | journal | get a better | Use your journal | | |
| | | | picture of your | to monitor your | | |
| | | | condition and | condition and | | |
| | | | triggers. | remind you of | | |
| | | | | your triggers. | | |
| | Journal | Carousel | Title: | Title: | Homepage, | Silent |
| | 02 | card, first | Let's add to your | Don't forget | carousel | push |
| | | time on the | journal and | adding to your | card | |
| | | app for the | get insights on | journal will give | | |
| | | day | your condition | insights on your | | |
| | | | Body: | condition | | |
| | | | Use your journal | Body: | | |
| | | | to monitor your | Use your journal | | |
| | | | condition and | to monitor your | | |
| | | | understand your | condition and | | |
| | | | triggers. | remind you of | | |
| | | | | your triggers. | | |
| | Journal | Ask users | Title: | Title: | Notification | Batched |
| | 03 | how they're | How are you | How are you | center | |
| | | doing, if no | doing today? | doing today? | | |
| | | journal | Body: | Body: | | |
| | | entry has | Hey [Denise], | Hey [Denise], | | |
| | | happened | looks like you | don't forget to | | |
| | | in the past | haven't added a | add a journal | | |
| | | three | journal entry in | entry to monitor | | |
| | | weeks | the past three | your condition | | |
| | | | weeks. | and remind you | | |
| | | | | of your triggers. | | |
| Journal: | Journal | Carousel | Title: | Title: | Homepage, | Silent |
| Follow up | 04 | card, follow | Any improvement | Any improvement | carousel | push |
| from flare | | up from a | with your flare | with your flare | card | |
| up | | prior | up? | up? | | |
| | | journal | Body: | Body: | | |
| | | entry from | Use your journal | Use your journal | | |
| | | previous | to monitor any | to monitor any | | |
| | | day | change in your | change in your | | |
| | | | condition since | condition since | | |
| | | | your initial | your initial | | |
| | | | incident. | incident. | | |
| | Journal | Follow up | Title: | Title: | Notification | Batched |
| | 05 | from a prior | Any improvement | Add a new | center | |
| | | journal | with your flare | journal entry | | |
| | | entry from | up? | Body: | | |
| | | a previous | Body: | Use your journal | | |
| | | day | Use your journal | to monitor any | | |
| | | | to monitor any | change in your | | |
| | | | change in your | condition since | | |
| | | | condition since | your initial | | |
| | | | your initial | incident. | | |
| | | | incident. | | | |
| | Journal | No recent | Title: | Title: | Notification | Batched |
| | 06 | journal | You haven't | You haven't | center | |
| | | entry | added a new | added a new | | |
| | | | journal entry | journal entry | | |
| | | | recently | recently | | |
| | | | Body: | Body: | | |
| | | | Regular journal | Regular | | |
| | | | entries will help | journaling will | | |
| | | | you and your | help you keep an | | |
| | | | doctor | ongoing record of | | |
| | | | understand how | how you're | | |
| | | | you're managing | managing your | | |
| | | | your [asthma/ | [asthma/atopic | | |
| | | | atopic dermatitis] | dermatitis]. | | |
| | | | while on [drug | | | |
| | | | name] | | | |
| Content: | Content | Injection | Title: | N/A | Notification | Push |
| Branded | 01 | training | Need a review of | (not first time | center | notification |
| content | | video, new | how to inject | user) | | |
| | | app user, | properly? | Title: | | |
| | | first time | Body: | Need an injection | | |
| | | user | The [drug name] | refresher? | | |
| | | | injection training | Body: | | |
| | | | video for your | Watch the [drug | | |
| | | | treatment is | name] injection | | |
| | | | always available | training video if | | |
| | | | to you in the app | you need that | | |
| | | | | quick refresher. | | |
| | Content | Upcoming | Title: | Title: | Notification | Batched |
| | 02 | injection, in | Let's get you | Need an injection | center | |
| | | the | ready for your | refresher? | | |
| | | adoption | injection in 2 | Body: | | |
| | | phase of | days | Review the step | | |
| | | [drug | Body: | by step guide if | | |
| | | name], 2 | Review the step | you want to get a | | |
| | | days prior | by step guide for | refresher on | | |
| | | to | your [drug name] | injection | | |
| | | scheduled | treatment and | guidance. | | |
| | | injection | become more | | | |
| | | | confident in | | | |
| | | | managing your | | | |
| | | | treatment routine. | | | |
| | Content | Upcoming | Title: | Title: | Notification | Push |
| | 03 | injection, in | Let's get ready | Your [drug name] | center | notification |
| | | the | for your [drug | dose is today | | |
| | | adoption | name] dose | Body: | | |
| | | phase of | Body: | If you need a | | |
| | | [drug | While your [drug | review of the | | |
| | | name], day | name] is getting | injection | | |
| | | of | to room | instructions, the | | |
| | | scheduled | temperature, why | step by step | | |
| | | injection | not review the | guide is available | | |
| | | | step by step | | | |
| | | | guide? | | | |
| | Content | New app | Title: | N/A | Notification | Batched |
| | 04 | user, in the | Hear how [drug | | center | |
| | | adoption | name] has | | | |
| | | phase of | helped others in | | | |
| | | [drug | a similar situation | | | |
| | | name] | to you. | | | |
| | | | Body: | | | |
| | | | Watch videos | | | |
| | | | from other [drug | | | |
| | | | name] users on | | | |
| | | | how the | | | |
| | | | treatment has | | | |
| | | | helped them get | | | |
| | | | their [asthma/ | | | |
| | | | atopic dermatitis] | | | |
| | | | under control. | | | |

In an example, a number of the notifications are required for offline operation, particularly notification names Treatments 06-09, Missed 01-03, Journal 01-06, Content 01-03.

There may be a prerequisite for at least some of the notifications to be shown. For example, for notification Treatment 02 a prerequisite may be that the user is already signed up to a proprietary Patient Support Program, whereas for notification Treatment 03 a prerequisite may be that the user is not signed up to the Program. A user may also indicate that they don't need or wish to see a particular notification and, in that case, the relevant notification will not be shown. For example, for notification Treatment 04, if the user has indicated that they don't need a step-by-step guide by toggling the appropriate option in the notification options (see FIG. 5a), this notification will not be shown. For some notifications the communicated action may also be dependent on a user's preference. For example, this may be the case for notification Treatment 07.

It will be appreciated that the desired outcome or action(s) following the various notifications will vary, for example from book a PSP call to review injection guide. Notification Treatment 06 may trigger a timer countdown relating to the time required for the medicament to warm up to room temperature. Following the notification for Treatment 07, once the user has indicated that the treatment is complete, for example, the injection may be logged. In response to notification Missed 01, the outcome/action may be that the user has already taken the dose, they just took the dose out of the fridge, remind me later. The outcome from notification Appointment 02 to 05 may be to generate a report or for notification Journal 01 to 06 to add a journal entry.

TABLE 2

| | Notification | | Notification | Notification |
|---|---|---|---|---|
| Purpose | name | Logic and Outcomes/Actions | point | Type |
| Prescription: | Prescription | Prescription is due to end in 1(?) | Notification | Push notification |
| Renewing | 01 | month, remind user to speak to | center | |
| prescription | | prescribing doctor; optionally | | |
| | | provide list of documents that are | | |
| | | relevant to continuing prescription, | | |
| | | if any | | |
| | Prescription | Prescription is due to end in 3 | Notification | Push notification |
| | 02 | weeks, follow up to Prescription | center | |
| | | 01. Confirm with user if they've | | |
| | | scheduled an appointment with | | |
| | | doctor or have spoken to them. | | |
| | | This information may be sent to a | | |
| | | Patient Support Program (if signed | | |
| | | up). | | |
| | Prescription | Prescription is due to end in 2 | Notification | Push notification |
| | 03 | weeks, follow up to Prescription | center | |
| | | 02. Confirm with user if they've | | |
| | | scheduled an appointment with | | |
| | | doctor or have spoken to them. | | |
| | | This information may be sent to a | | |
| | | Patient Support Program. | | |
| | Prescription | Prescription is due to end in 1 | Notification | Push notification |
| | 04 | weeks, follow up to Prescription | center | |
| | | 03. Confirm with user if they've | | |
| | | scheduled an appointment with | | |
| | | doctor or have spoken to them. | | |
| | | This information may be sent to a | | |
| | | Patient Support Program (if signed | | |
| | | up). | | |
| | Prescription | If document needs to be submitted | Notification | Push notification |
| | 05 | for prescription/insurance | center | |
| | | purposes to renew the | | |
| | | prescription. Remind user to | | |
| | | submit document. | | |
| Insurance | Insurance | Insurance coverage has been | Notification | Batched |
| | 01 | approved | Center | |
| | Insurance | Insurance status update | Notification | Batched |
| | 02 | | Center | |
| | Insurance | Evaluating user for patient | Notification | Batched |
| | 03 | assistance program | Center | |
| | Insurance | Checking with users around | Notification | Batched |
| | 04 | changes to their insurance - quick | Center | |
| | | survey question | | |
| | Insurance | Ask user to submit insurance card | Notification | Batched first |
| | 05 | or indicate that they don't have | Center | time |
| | | one yet. | | Push notification |
| | | | | if user doesn't |
| | | | | respond in 24 |
| | | | | hours |
| | Insurance | Submit insurance card, follow up | Notification | Batched first |
| | 06 | to Insurance 05 if user selected I | Center | time |
| | | don't have it yet. Pushed to user | | Push notification |
| | | the following week. | | if user doesn't |
| | | | | respond in 48 |
| | | | | hours |
| Nurse | Nurse 01 | A Patient Support Program nurse | Notification | Push notification |
| | | tried contacting user. | Center | (if a Patient |
| | | Suggest user call the Patient | | Support |
| | | Support Program or Request call | | Program is |
| | | back. | | open) |
| | | | | Batched (if out |
| | | | | of hours) |
| | Nurse 02 | A Patient Support Program | Notification | Batched first |
| | | received Medicare Part D re- | Center | time |
| | | enrolment form, will reach out if | | Push notification |
| | | additional information is needed | | if user doesn't |
| | | | | respond in 48 |
| | | | | hours |
| Documents | Document | Issue with Medicare Part D Patient | Notification | Batched first |
| | 01 | Assistance Program form | Center | time |
| | | | | Push notification |
| | | | | if user doesn't |
| | | | | respond in 48 |
| | | | | hours |
| | Document | Asking for submission of Medicare | Notification | Batched first |
| | 02 | Part D Patient Assistance | Center | time |
| | | Program form | | Push notification |
| | | | | if user doesn't |
| | | | | respond in 48 |
| | | | | hours |
| | Document | Additional documentation request, | Notification | Batched first |
| | 03 | requiring three documents: | Center | time |
| | | - [year] social security awards | | Push notification |
| | | letter | | if user doesn't |
| | | - [year] federal tax returns | | respond in 48 |
| | | - Last 3 months of bank | | hours |
| | | statements | | |
| | Document | proof of income | Notification | Batched first |
| | 04 | User may be prompted to submit | Center | time |
| | | proof of income or indicate that | | Push notification |
| | | they have already submitted the | | if user doesn't |
| | | document or request guidance on | | respond in 48 |
| | | what is required. | | hours |
| | Document | Upload Extra Help determination | Notification | Batched first |
| | 05 | letter | Center | time |
| | | (or ask user indicate that this | | Push notification |
| | | document has already been | | if user doesn't |
| | | submitted or request further | | respond in 48 |
| | | guidance) | | hours |
| | Document | A Patient Support Program | Notification | Batched first |
| | 06 | received Medicare Part D re- | Center | time |
| | | enrolment form, will reach out if | | Push notification |
| | | additional information is needed | | if user doesn't |
| | | | | respond in 48 |
| | | | | hours |
| | Document | A Patient Support Program | Notification | Batched first |
| | 07 | received proof of income, will | Center | time |
| | | reach out if additional information | | Push notification |
| | | is needed | | if user doesn't |
| | | | | respond in 48 |
| | | | | hours |
| | Document | A Patient Support Program | Notification | Batched first |
| | 08 | received Extra Help document, will | Center | time |
| | | reach out if additional information | | Push notification |
| | | is needed | | if user doesn't |
| | | | | respond in 48 |
| | | | | hours |
| | Document | Issue with Proof of Income | Notification | Batched first |
| | 09 | document, resubmit | Center | time |
| | | | | Push notification |
| | | | | if user doesn't |
| | | | | respond in 48 |
| | | | | hours |
| | Document | Issue with Extra Help document, | Notification | Batched first |
| | 10 | resubmit | Center | time |
| | | | | Push notification |
| | | | | if user doesn't |
| | | | | respond in 48 |
| | | | | hours |

The application may also receive data from external devices or from other applications running on the user device. For example, connected devices such as activity trackers and heart rate monitors may be paired with the application. The application may also gather and combine information from other programs such as environmental monitoring apps or weather forecasts. Some further notifications may be generated based on the information obtained from these external devices or services. For example, notifications about high pollen count, high air pollution, high UV index, high or low humidity or particular weather conditions may be generated on the basis of information obtained from environmental monitoring apps or weather forecasts.

The priority levels for these notifications may depend on the medical conditions that the particular user has. For example, if the user has Asthma, then notifications relating to high pollen count and high air pollution may have a high priority and be displayed as a push notifications. Whereas if the user has Atopic Dermatitis, but not Asthma, then these notifications may be displayed with a lower priority, such as a batch notifications or silent push, or not displayed at all.

Notifications may also be generated based on the user's activity levels, as determined by data from external devices such as smart watches and other activity trackers. For example, if the obtained data indicates that the user has had poor sleep, the notifications may be generated asking the user if they wish to create a journal entry relating to this, as shown in FIG. 4b. As such a notification is time sensitive (since it relates to recent user activity) it may be given a high priority and displayed as a push notifications. General notifications relating to environmental conditions which might exacerbate the user's medical conditions, such as certain weather conditions or high UV index, but which do not suggest some time sensitive action on the part of the user, may be displayed as batch notifications or silent push notifications.

The application may combine information from both activity trackers and environmental monitors. For example, the application may user a step count to determine at what time the user is taking a walk. This may be cross referenced with pollen and/or air pollution data from the environmental monitor to determine that the user regularly takes a walk when the pollen level and/or pollution levels are high. A notification may then be generated advising the user that this may be exacerbating or triggering their medical conditions and advise taking walks at a different time. This notification may be considered non time sensitive, since it relates to longer term behavioral change, and therefore displayed as a batch notification or a silent push notification, for example in the learning tab screen of the application.

The application may also be connected with other smart devices around the home, such as a smart fridge, a smart TV, a tablet computing device, a connected storage device for storing the user's medicament, or a connected sharps bin. Notifications may be generated based on the data received from these devices. For example, if the application is notified by a smart fridge that the door has been opened within the pre-defined window for the user to take their medicament, a notification may be generated asking the user if they wish to start a warm-up timer. Some medicaments must be stored in the fridge but brought up to room temperature to be comfortably injected. Generating a warm-up timer reminder at the time when the user opens the fridge door can help to remind the user of this and is a timely and useful reminder that the application can provide the warm-up timer for the user. Since this is a time sensitive notification, it may be displayed as a push notification.

As a further example, if the application receives data from a connected storage device, that the user has removed an injection device from the storage device, the application may generate a notification asking the user if they wish to view a step-by-step guide to self-injection. Since this is a time sensitive notification, it may be displayed as a push notification. Such a notification may be displayed the first time a user takes an injection device from the storage device, or the first few times, or every time. In another example, the notification may relate to ordering more medicament or renewing a prescription, if the data from the connected store device indicates that no more injection devices (or only one more injection device) remain in the storage device.

As a further example, if the application receives data from a connected smart bin indicating that the user has deposited an injection device into the sharps bin, the application may generate a notification asking the user if they wish to schedule their next injection. Such a notification may only be displayed if no injection schedule has been set in the application. In another example, the notification may ask the user if they wish to create a journal entry or if they wish to talk to a healthcare professional about their medical condition or treatment.

By utilizing data received from external devices and services in this way, the application can provide more timely and relevant notifications to a user which greatly increase the ability of the application to assist the user in managing their health conditions and can also improve user satisfaction and adherence with their prescribed medicament.

It can be seen that some of the notifications are providing the user with confirmation that an input such as a document has been received, whereas other notifications are prompting the user to perform an action.

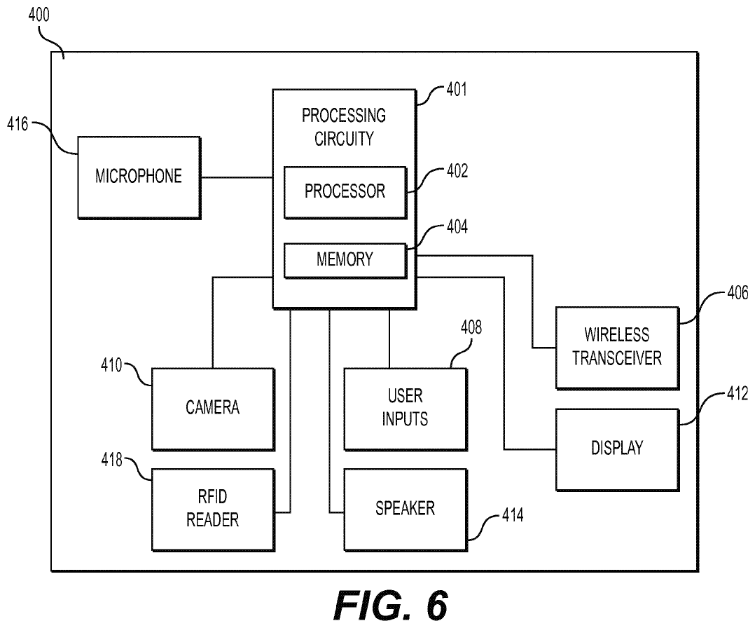

FIG. 6 illustrates schematically a user device 400 according to some embodiments. The user device 400 may be a mobile phone or tablet computer. The user device 400 is an example of a wireless communication device. The user device may alternatively be referred to as a communication device, computer, computing device or mobile device and is not necessarily associated with a single user. The user device 400 is configured to communicate wirelessly with a communications network using a wireless communication protocol, for example but not exclusively 3GPP LTE and/or New Radio (NR) or WiFi (IEEE 802.11). The user device 400 may also be configured to communicate using Bluetooth, NFC, Zigbee, Ultra Wideband, IrDa or similar. The communications network may comprise one or more network nodes. The communications network may further be connected via a core network and/or an intermediate network to a host computer (not shown) which may be embodied in the hardware and/or software of a standalone server, a cloud-implemented server or a distributed server. The user device 400 and the host computer may be configured to communicate data.

The user device 400 may comprise hardware that includes processing circuitry 401. The processing circuitry 401 may comprise a processor 402 and a memory 404. The user device 400 may also comprise a wireless transceiver 406, user inputs 408, a display 412, a camera 410, a microphone 416, and RFID reader 418 and a speaker 414. The wireless transceiver 406 may be configured to set up and maintain a wireless connection to a network node. The wireless transceiver 406 may comprise one or more radio transmitters and one or more radio receivers. The display may be a touch sensitive display and may be based on capacitive or resistive sensing technology. The processing circuitry 401 may for instance include a microprocessor, a Digital Signal Processor (DSP), Application Specific Integrated Circuit (ASIC), Field Programmable Gate Array (FPGA) or the like. The processor 402 may be configured to read and/or write from the memory 404. The memory 404 may comprise a volatile and/or nonvolatile memory, for example a cache, RAM (Random Access Memory) and/or ROM (Read-Only Memory) etc.

The user device 400 may comprise software which is stored, for example, in memory 404. The software may be executable by the processing circuitry 401. The software may include an application. In some embodiments the host computer may communicate with the application. The application may request data from the host computer and/or provide user data to the host computer.

The processing circuitry 401 may be configured to perform or cause to be performed any of the methods described herein. In some embodiments, the software/program may include instructions that, when executed by the processing circuitry 401 cause the processing circuitry 401 to perform the methods described herein.

The memory 404 may comprise both a program memory storing program code (e.g. software or firmware) and main memory storing data. The processing circuitry 401 may be configured to execute the program code stored in the program memory and to read, write and delete data from the main memory. In some embodiments, the program code may be an application which can be downloaded and installed on the user device 400. The application may be a disease and treatment management and tracking tool for use by patients. The program memory may for instance be a Read-Only Memory (ROM), and the main memory may for instance be a Random Access Memory (RAM).

The user device 400 comprises one or more user inputs 408, for example a touchscreen, keypad or keyboard, accelerometer or gyroscope, mouse or microphone 416 for receiving voice commands. User device 400 may also comprise a camera 410 configured to capture images of a user and images of labels, codes and the like visible on the medicament administration devices, packaging or storage solutions. The user device 400 may be configured to scan medicament administration devices (such as an injection device or inhaler) using a scanning device. The scanning device may refer to either the camera 410 or RFID reader 418. The term “scanning” as used in relation to the user device 400 may refer to use of either of these components to read information provided externally or internally on medicament administration devices.

The notification scheme outlined above provides some key advantages. Predictability: there is a consistent approach to how notifications are presented to users, prioritizing urgent notifications and clearly indicating any actions required of users. For example, any notification which requires the user to take some time sensitive action will be sent as a push notification and will indicate the action which the user needs to take. The user is not sent a push notification for all notifications and learns to associate push notifications from the app as being of high importance and requiring action on their part. Reliability: the notifications are consistent and reliable and therefore demonstrate dependability to the user. The user's learn to trust that the application will guide them through their treatment and present the right resources at the right time. Context aware: the notifications are contextually aware (i.e. take into account the user's situation, preferences and level of experience). Notifications are communicated at the appropriate time with the appropriate level of priority.

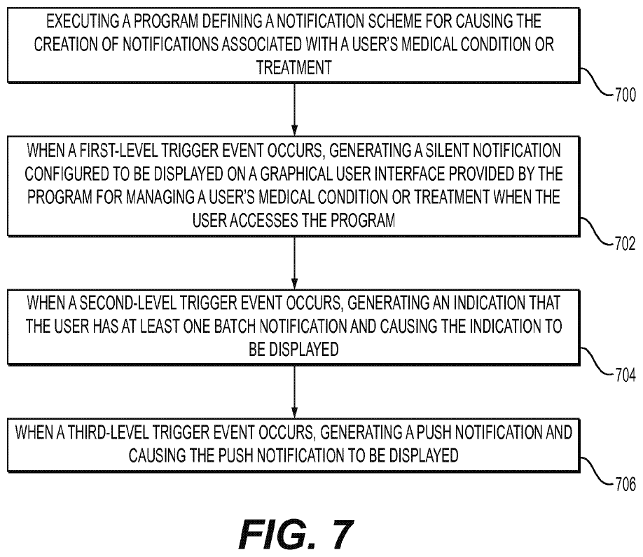

FIG. 7 is a flow chart illustrating the steps of a computer implemented method. At step 700, the method requires executing a program defining a notification scheme for causing the creation of notifications associated with a user's medical condition or treatment. The program is the previously described application for managing a user's medical condition or treatment. The program is executed at least partially on a user device and the notifications are displayed on a display of the user device.

At step 702 the method requires, when a first-level trigger event occurs, generating a silent notification configured to be displayed on a graphical user interface provided by the program for managing a user's medical condition or treatment when the user accesses the program. The silent notification may be displayed on a home screen of the program for managing a user's medical condition. In some other embodiments the silent notification may be displayed on a treatment page of the program for managing a user's medical condition in addition to the home screen, depending on the subject matter of the notification.

At step 704 the method requires, when a second-level trigger event occurs, generating an indication that the user has at least one batch notification and causing the indication to be displayed. Interacting with the batch notification may open a notification center where the content of the individual notifications can be seen and further interacted with. The batch notification may be displayed on a graphical user interface provided by an operating system of a user device. This graphical user interface may be the lock screen of the user device.

At step 706 the method requires, when a third-level trigger event occurs, generating a push notification and causing the push notification to be displayed. Interacting with the push notification may cause the program for managing a user's medical condition or treatment to open and navigate to a user interface where action related to the subject of the push notification can be taken. The push notification may be displayed on a graphical user interface provided by an operating system of a user device. This graphical user interface may be the lock screen of the user device.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body. The drugs or medicaments contained in the drug delivery devices as described herein can be used for the treatment and/or prophylaxis of many different types of medical disorders.

The term “antibody”, as used herein, refers to an immunoglobulin molecule or an antigen-binding portion thereof. Examples of antigen-binding portions of immunoglobulin molecules include F (ab) and F(ab′) 2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′) 2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6R mAb (e.g., Sarilumab), and anti IL-4R mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1.-20. (canceled)

21. One or more non-transitory computer-readable storage media storing instructions that, when executed by a computer, cause the computer to:
execute a program for managing a medical condition or treatment of a user, the program defining a notification scheme for causing notifications associated with the medical condition or treatment of the user;
when a first-level trigger event occurs, generate a silent notification configured to be displayed on a graphical user interface provided by the program for managing the medical condition or treatment of the user when the user accesses the program;
when a second-level trigger event occurs, generate an indication that the user has at least one batch notification and cause the indication to be displayed; and
when a third-level trigger event occurs, generate a push notification and cause the push notification to be displayed,
wherein the indication that the user has at least one batch notification and the push notification are caused to be displayed on another graphical user interface provided by an operating system of a user device comprising the computer.

22. The one or more non-transitory computer-readable storage media of claim 21, wherein the other graphical user interface provided by the operating system of the user device is a lock screen of the user device.

23. The one or more non-transitory computer-readable storage media of claim 21, wherein the graphical user interface provided by the program for managing the medical condition or treatment of the user is a home screen of the program for managing the medical condition or treatment.

24. The one or more non-transitory computer-readable storage media of claim 21, wherein the second-level trigger event has a higher priority than the first-level trigger event, and the third-level trigger event has a higher priority than the second-level trigger event.

25. The one or more non-transitory computer-readable storage media of claim 21, wherein the at least one batch notification defines a threshold time for interacting with the at least one batch notification and, if the at least one batch notification is not interacted with within the threshold time, the computer is caused to generate the push notification relating to the at least one batch notification.

26. The one or more non-transitory computer-readable storage media of claim 21, wherein content of at least one of the silent notification or the push notification varies depending on an experience level of the user.

27. The one or more non-transitory computer-readable storage media of claim 21, wherein the push notification reminds the user to remove a medicament delivery device from a low temperature environment, and in response to the user interacting with the push notification, the computer is caused to start a timer for allowing a medicament in the medicament delivery device to reach room temperature.

28. The one or more non-transitory computer-readable storage media of claim 21, wherein the first-level trigger event, the second-level trigger event or the third-level trigger event comprises a time remaining before a next medicament administration is due.

29. The one or more non-transitory computer-readable storage media of claim 21, wherein the first-level trigger event, the second-level trigger event, or the third-level trigger event comprises a time remaining before an appointment or call.

30. The one or more non-transitory computer-readable storage media of claim 21, wherein the first-level trigger event, the second-level trigger event, or the third-level trigger event comprises a prompt to create a journal entry regarding disease symptoms and/or overall health.

31. The one or more non-transitory computer-readable storage media of claim 21, wherein the first-level trigger event, the second-level trigger event, or the third-level trigger event comprises a time remaining before a prescription period is due to end.

32. A computer-implemented method comprising:
executing a program for managing a medical condition or treatment of a user, the program defining a notification scheme for causing notifications associated with the medical condition or treatment;
when a first-level trigger event occurs, generating a silent notification configured to be displayed on a graphical user interface provided by the program for managing the medical condition or treatment of the user when the user accesses the program;
when a second-level trigger event occurs, generating an indication that the user has at least one batch notification and cause the indication to be displayed; and
when a third-level trigger event occurs, generating a push notification and cause the push notification to be displayed,
wherein the indication that the user has at least one batch notification and the push notification are caused to be displayed on another graphical user interface provided by an operating system of a user device.

33. The computer-implemented method of claim 32, wherein the other graphical user interface provided by the operating system of the user device is a lock screen of the user device and wherein the graphical user interface provided by the program for managing the medical condition or treatment of the user is a home screen of the program for managing the medical condition or treatment.

34. The computer-implemented method of claim 32, wherein the at least one batch notification defines a threshold time for interacting with the at least one batch notification and, if the at least one batch notification is not interacted with within the threshold time, the method further comprises generating the push notification relating to the at least one batch notification.

35. The computer-implemented method of claim 32, wherein the push notification reminds the user to remove a medicament delivery device from a low temperature environment, and
wherein the method further comprises, in response to the user interacting with the push notification, starting a timer for allowing a medicament in the medicament delivery device to reach room temperature.

36. The computer-implemented method of claim 32, wherein the first-level trigger event, the second-level trigger event or the third-level trigger event is each selected from a group comprising (i) a time remaining before a next medicament administration is due, (ii) a time remaining before an appointment or call, (iii) a prompt to create a journal entry regarding disease symptoms and/or overall health, and (iv) comprises a time remaining before a prescription period is due to end.

37. A user device comprising one or more processors and one or more storage devices storing instructions that, when executed by the one or more processors, cause the one or more processors to:
execute a program for managing a medical condition or treatment of a user, the program defining a notification scheme for causing notifications associated with the medical condition or treatment;
when a first-level trigger event occurs, generate a silent notification configured to be displayed on a graphical user interface provided by the program for managing the medical condition or treatment of the user when the user accesses the program;
when a second-level trigger event occurs, generate an indication that the user has at least one batch notification and cause the indication to be displayed; and
when a third-level trigger event occurs, generate a push notification and cause the push notification to be displayed,
wherein the indication that the user has at least one batch notification and the push notification are caused to be displayed on another graphical user interface provided by an operating system of the user device.

38. The user device of claim 37, wherein the other graphical user interface provided by the operating system of the user device is a lock screen of the user device and wherein the graphical user interface provided by the program for managing the medical condition or treatment of the user is a home screen of the program for managing the medical condition or treatment.

39. The user device of claim 37, wherein the at least one batch notification defines a threshold time for interacting with the at least one batch notification and, if the at least one batch notification is not interacted with within the threshold time, the user device is configured to generate the push notification relating to the at least one batch notification.

40. The user device of claim 37, wherein the push notification reminds a user to remove a medicament delivery device from a low temperature environment, and in response to the user interacting with the push notification, the user device is configured to start a timer for allowing a medicament in the medicament delivery device to reach room temperature.
